(19) United States
(12) Patent Application Publication
(10) Pub. No.: US 2025/0281377 A1
(43) Pub. Date: Sep. 11, 2025

(54) TOPICAL HYDRATION COMPOSITIONS

(71) Applicant: GALDERMA HOLDING SA, ZUG (CH)

(72) Inventors: Claudia Victoria PEDRAZA NOCUA, Montreal (CA); Trong Dat NGO, Laval (CA); Margaux Julia LOUVEL, SURESNES (FR); Virginie LE NOËL, Lugrin (FR); Michelle Elizabeth GREEN, Carrollton, TX (US)

(21) Appl. No.: 18/858,556

(22) PCT Filed: Apr. 17, 2023
(86) PCT No.: PCT/IB2023/053922

§ 371 Date: Oct. 21, 2024

(30) Foreign Application Priority Data
Apr. 22, 2022 (EP) 22169474.8
Jul. 8, 2022 (EP) 22183763.6

(51) Int. Cl. A61K 8/49; A61K 8/04; A61K 8/24; A61K 8/34; A61K 8/35; A61K 8/365; A61K 8/42; A61K 8/44; A61K 8/60; A61K 8/67; A61K 8/73; A61K 8/81; A61K 8/88; A61K 8/891; A61K 8/9789; A61Q 19/00

(52) U.S. Cl. CPC A61K 8/4946; A61K 8/042; A61K 8/24; A61K 8/345; A61K 8/35; A61K 8/365; A61K 8/42; A61K 8/44; A61K 8/602; A61K 8/675; A61K 8/732; A61K 8/733; A61K 8/735; A61K 8/8152; A61K 8/8158; A61K 8/88; A61K 8/891; A61K 8/9789; A61Q 19/005; A61Q 19/007; A61K 2800/30; A61K 2800/522

(57) ABSTRACT

The present invention includes compositions for skin hydration, moisturization, and repair. The compositions provide unique combinations of different components, including hydrating agents, moisturizing agents and polymers facilitating sustained hydration. The compositions have different uses, including skin hydration, skin moisturization, the treatment of dry skin and/or the treatment of sensitive skin. Preferred compositions are an oil-free gel providing long lasting hydration.

### CROSS-REFERENCE TO RELATED APPLICATION

This application claims priority to EP Patent Application No. 22183763.6, filed on Jul. 8, 2022, and to EP Patent Application No. 22169474.8, filed on Apr. 22, 2022, the disclosures of which are incorporated herein by reference in their entirety.

### FIELD OF THE INVENTION

The present invention generally relates to topical compositions. The compositions can be used, for example, for skin hydration, skin moisturization, treatment of dry skin and/or treatment of sensitive skin.

### BACKGROUND OF THE INVENTION

Dry skin and sensitive skin are common dermatologic issues. Symptoms of dry skin include dehydration, flakes, cracks, pruritus, and peeling. Treatment of dry skin includes moisturizing and hydrating agents.

Similar to dry skin, sensitive skin causes a range of uncomfortable symptoms. Sensitive skin is clinically defined by characteristic sensory perceptions including tightness, abnormal stinging, burning, tingling, pain, and pruritus (Misery et al. JEADV, 2016, 30: 2-8). Patients with sensitive skin typically present without visible skin lesions. Several studies have suggested a link between sensitive skin and a disruption of the epidermal barrier function, resulting in the perception of skin discomfort (Id.). A weak epidermal barrier facilitates the penetration of irritants or allergens, fails to adequately protect nerve endings, and increases trans-epidermal water loss (TWEL).

There is a link between dry skin and skin sensitivity. Dry skin can experience an increase in skin sensitivity due to its compromised moisture barrier. Proper skin hydration levels help the skin to be resilient and strong. Topical compositions used to treat dry or sensitive skin seek to improve the overall quality of the skin by increasing the skin's water content and sealing moisture within.

### BRIEF SUMMARY OF THE INVENTION

The present invention includes compositions for skin hydration, moisturization, and skin repair. The compositions provide unique combinations of different components, including hydrating agents, moisturizing agents and polymers facilitating sustained hydration. The compositions have different uses, including skin hydration, skin moisturization, the treatment of dry skin and/or the treatment of sensitive skin. Preferred compositions are an oil-free gel providing long lasting hydration.

Thus, a first aspect of the present invention describes a topical composition comprising;
(i) 0.05% to 0.6% by weight allantoin;
(ii) 1.0% to 5.0% by weight Combination A comprising 12% to 22% glycerin, 5% to 15% trehalose, 5% to 15% urea, 1% to 5% serine, 0.5% to 4% pentylene glycol, 0.10% to 2% algin or a cosmetically acceptable salt thereof, 0.1% to 0.5% glyceryl polyacrylate, 0.2% to 0.8% caprylyl glycol, 0.1% to 1.0% hyaluronic acid or a cosmetically acceptable salt thereof, 0.1% to 1.0% pullulan, and water;
(iii) 0.005% to 0.5% by weight hydrolyzed hyaluronic acid or a cosmetically acceptable salt thereof,
(iv) 0.05% to 1.0% by weight polyglutamic acid or a cosmetically acceptable salt thereof, and
(v) water.

The amount of water varies depending upon the total amount of agents present. In certain embodiments the total amount of water for the topical composition, including that present in Combination A, is between 60% and 85% by weight. A variety of additional agents may be present including one or more solvents, skin protecting agents, humectants, emollients, preservatives, skin conditioners, anti-inflammatory agents, emulsion agents, absorbents, buffering agents and pH adjustors. A particular agent may have more than one type of activity.

Another aspect of the present invention is directed to a method of treating the skin in a subject comprising applying to the skin an effective amount of the composition described herein to hydrate the skin, moisturize the skin, treat dry skin, and/or treat sensitive skin.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 illustrates the evolution of cutaneous hydration rate in subjects treated with a single application of Product A over 72 hours (t=time).

### DETAILED DESCRIPTION OF THE INVENTION

The present invention provides combinations of different components, including different hydrating and moisturizing agents and polymers facilitating sustained hydration. As illustrated in the examples below, uses of the compositions include skin hydration, skin moisturization, the treatment of dry skin and/or the treatment of sensitive skin. Preferred compositions are an oil-free gel providing long lasting hydration.

A first aspect of the present invention describes a composition comprising;
(i) 0.05% to 0.6% by weight allantoin;
(ii) 1.0% to 5.0% by weight Combination A comprising 12% to 22% glycerin, 5% to 15% trehalose, 5% to 15% urea, 1% to 5% serine, 0.5% to 4% pentylene glycol, 0.10% to 2% algin or a cosmetically acceptable salt thereof, 0.1% to 0.5% glyceryl polyacrylate, 0.2% to 0.8% caprylyl glycol, 0.1% to 1.0% hyaluronic acid or a cosmetically acceptable salt thereof, 0.1% to 1.0% pullulan, and water; in an embodiment, Combination A further comprises 0.05% to 0.4% disodium phosphate and 0.005% to 0.04% potassium phosphate;
(iii) 0.005% to 0.5% by weight hydrolyzed hyaluronic acid or a cosmetically acceptable salt thereof,
(iv) 0.05% to 1.0% by weight polyglutamic acid or a cosmetically acceptable salt thereof, and
(v) water.

The total amount of water varies depending upon the total amount of agents present. In certain embodiments the total amount of water, including that present in Combination A, is between 60% and 85% by weight. In certain embodiments, the total amount of water is between 65% to 83%, 70% to 80%, or about 76% by weight.

In those cases where water is referred to without providing an indicated amount, embodiments are provided where (1) the indicated amount is that amount needed to provide 100% of the components and (2) lesser amount of water allowing for additional agents which, together with water, provide 100%.

In different embodiments, the weight percent of allantoin is 0.05% to 0.6%, 0.05% to 0.3% or about 0.3%; the weight percent of Compound A is 1.0% to 5.0% or about 2.0%; the weight percent of hydrolyzed hyaluronic acid is 0.005% to 0.5%, 0.05% to 0.5%, 0.1% to 0.5%, or about 0.01%, about 0.05%, about 0.1%, or about 0.2%; and/or the weight percent of polyglutamic acid or a cosmetically acceptable salt thereof is 0.05% to 1.0%, 0.1% to 0.5%, or about 0.1%. In further embodiments, the polyglutamic acid has a molecular weight of 30,000 Daltons (30 kDa) to 150 kDa, or 50 kDa to 100 kDa.

Combination A provides a molecular patch based on the film-forming natural biopolymers hyaluronic acid or a cosmetically acceptable salt thereof, algin or a cosmetically acceptable salt thereof and pullulan. The biopolymers carry highly concentrating substances such as trehalose, glycerin, urea and L-serine providing for hydration and/or moisturization. In certain embodiments, Combination A is PatcH₂O®; or comprises by weight percent:
about 17.5% glycerin;
about 10% trehalose;
about 10% urea;
about 3% serine;
about 2% pentylene glycol;
about 0.5% algin or a cosmetically acceptable salt thereof, having a molecular weight of 100 to 550 kDa, preferably sodium alginate;
about 0.3% glyceryl polyacrylate;
about 0.5% caprylyl glycol;
about 0.25% hyaluronic acid or a cosmetically acceptable salt thereof, having a molecular weight of 250 to 450 kDa, preferably sodium hyaluronate;
about 0.25% pullulan, wherein the pullulan has a molecular weight of 100 to 250 kDa;
about 0.1% disodium phosphate;
about 0.02% potassium phosphate; and water.

Without being limited to any particular theory, the biopolymers provide for sustained release of different agents, including those provided in Combination A, and also other agents that may be present in the composition. The allantoin increases the capacity of corneocytes to bind water, reinforcing the skin's natural protective barrier and improves moisture retention. Polyglutamic acid benefits include increasing skin moisture, protecting the skin, helping maintain healthy skin pH, and reducing degradation of hyaluronic acid by inhibiting hyaluronidase.

Combinations of hyaluronic acid or a cosmetically acceptable salt thereof, alginic acid or a cosmetically acceptable salt thereof, and pullulan, along with examples of mixtures containing such combinations are described in US Patent Application No. 2015/0209264 (“US 20150209264” hereby incorporated by reference herein in its entirety).

A cosmetically acceptable “salt” of a compound is a non-toxic salt suitable for application to human skin. Reference to a percentage of compound or a cosmetically acceptable salt includes the indicated compound (e.g., in acid form), a salt of the compound, and combinations of compound and the salt form. Reference to a particular salt form indicates that the salt form predominates, but allows for small amounts of the acid form that may be provided due to an equilibrium reaction. Similarly, reference to a particular acid form allows for small amounts of the salt form that may be provided by an equilibrium reaction.

In different embodiments, the alginic acid or a cosmetically acceptable salt thereof has a molecular weight of 10 to 600 kDa, of 30 to 550 kDa, or preferably of 100 to 550 kDa. Examples of salts formed with alginic acid include ammonium alginate, sodium alginate, calcium alginate, magnesium alginate, sodium sulphate alginate and potassium alginate. (See, e.g., US 20150209264).

In different embodiments, the hyaluronic acid or cosmetically acceptable salt thereof has a molecular weight of greater than 20 kDa, or has a molecular weight of 50 to 800 kDa or 250 to 450 kDa. Examples of salts formed with hyaluronic acid are hydrolyzed calcium hyaluronate, hydrolyzed sodium hyaluronate, potassium hyaluronate, sodium hyaluronate, sodium sulphated hyaluronate and mixtures thereof (See, e.g., US 20150209264).

The composition can be supplemented with a variety of different agents. Additional agents that may be present include one or more solvents, skin protecting agents, humectants, emollients, preservatives, anti-inflammatory agents, emulsion agents, absorbents, buffering agents, and pH adjusting agents. A particular agent may have more than one type of activity.

A solvent is a substance used to dissolve other substances. Examples of cosmetic solvents include water, glycerin, silicones, alcohol and Symdiol® 68. Preferred solvents are oil-free.

Humectants are substances that attract water. Examples of cosmetic humectants include gelatin, glycerin, glycerol, erythritol, arabitol, xylitol, ribitol, hyaluronic acid, panthenol, propylene glycol, butylene glycol, hydroxy acids, glycolic acid, lactic acid, sodium pyrrolidine carboxylic acid, sorbital, urea, Aquaxyl®, hydrolyzed hyaluronic acid, polyglutamic acid and PatcH₂O®.

Emollients are substances that improve skin barrier function. Emollients can soften and condition skin by filling in the gaps between cells/skin flakes. Examples of emollients include Symdiol® 68, fatty alcohols, cholesterol, ceramide, silicon, undecane, tridecane, dicaprylyl ether, and decyl oleate.

Preservatives are substances that prevent or retard bacterial growth. Examples of preservatives include antioxidants (e.g., hydroxyacetophenone), aldehydes (e.g., formaldehyde, DMDM hydantoin, imadozolidinyl urea, and diazolidinyl urea), glycol ethers (e.g., phenoxyethanol and caprylyl glycol), parabens (e.g., methylparaben, ethylparaben, propylparaben, butylparaben, and isobutylparaben), methylisothiazolinone, benzoic acid, sorbic acid, levulinic acid, anisic acid, SymSave® H and Symdiol® 68.

Anti-inflammatory agents reduce inflammation. Examples of anti-inflammatory agents include aloe vera, witch hazel, niacinamide, chamomile, and Phytessence® Blue Daisey.

Emulsion agents are used as adjuvants for combining non-miscible fluids into an emulsion. Examples of emulsion agents include Pemulen® TR-1, Pemulen® TR-2, carbomer, acrylates/C10-30 alkyl acrylate cross polymer, sodium lauryl sulphate benzalkonium chloride, behentrimonium methosulfate, cetearyl alcohol, stearic acid, glyceryl stearate and ceteareth-20.

Absorbents are substances having the capacity to absorb or soak up water. Examples of absorbents include distarch phosphate, Avena stiva starch, modified corn starch, aluminum starch octenylsuccinate, kaolin, corn starch, oat starch, cyclodextrin, talc, and zeolite.

Buffering agents and pH adjustors are substances that help provide for a particular pH or pH range. Buffering agents also help maintain a particular pH or pH range. Examples of buffering agents and pH adjustors include sodium hydroxide, potassium hydroxide, lactic acid, citric acid, ascorbic acid, adipic, ammonium oxide, calcium oxide, disodium phosphate, triethanolamine, and disodium phosphate/potassium phosphate. In the different embodiments, the composition has a pH range of 4 to 6.5, 4 to 7, 4 to 6.5, 4.5 to 6.5 or 4 to 6.

Reference to “about” indicates a difference from the stated amount of up to 20%. The difference can be greater or less than the stated amount. For example, a 20% difference of 10 provides a range of 8-12. In different embodiments, about refers to a difference of up to 10%, a difference up to 5%, or no difference.

All numerical values or numerical ranges include the initial value, the final value, integers within such ranges and fractions of the values or the integers within ranges, unless the context clearly indicates otherwise. Thus, to illustrate, reference to 50% to 75% includes 50% 75% and all percentage in between.

Unless expressly indicated otherwise, all percentages of composition components including particular agents, combination of agents, and agents making up a combination are weight percentages.

Reference to a subject indicates a human, and reference to skin indicates human skin.

The singular forms “a,” “an,” and “the” include plural reference, unless the context clearly dictates otherwise.

As used herein, the conjunctive term “and/or” between multiple recited elements is understood to encompass both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning, and therefore satisfy the requirement of the term “and/or”. Concurrent applicability of more than one of the options is also understood to fall within the meaning of the term “and/or”.

Unless clearly indicated otherwise by the context employed, the terms “or” and “and” have the same meaning as “and/or”.

Reference to terms such as “including”, “for example”, “e.g.”, “such as” followed by different members or examples, are open-ended descriptions where the listed members or examples are illustrative and other member or examples can be provided or used.

Reference to “comprise”, and variations such as “comprises” and “comprising”, used with respect to an element or group of elements, is open-ended and does not exclude additional unrecited elements or method steps. Terms such as “including”, “containing” and “characterized by” are synonymous with comprising. In the different aspects and embodiments described herein, reference to an open-ended term such as “comprising” can be replaced by the terms “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application apply throughout the present application.

Unless defined otherwise, all technical and scientific terms used herein have the same meaning commonly understood to one of ordinary skill in the art to which this invention pertains.

The description has been separated into various sections and paragraphs, and provides various embodiments. These separations should not be considered as disconnecting the substance of a paragraph or section or embodiments from the substance of another paragraph or section or embodiment. The provided descriptions have broad application and encompasses all the combinations of the various sections, paragraphs and sentences that can be contemplated. The discussion of any embodiment is meant only to be exemplary and is not intended to suggest the scope of the disclosure, including the claims (unless otherwise provided in the claims), is limited to these examples.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

### I. ADDITIONAL EMBODIMENTS

Examples of additional embodiments including particular combinations of agents are provided in the present section. In some cases, terms such as emollient, humectant, absorbent, emulsion, buffering agent and pH adjuster are used for an agent or combination of agents. Reference to terms such as emollient, humectant, absorbent, emulsion, buffering agent and pH adjuster with respect to a particular agent or group of agents provide an expected, but not required, activity. In addition, a particular agent can have more than one type of activity.

In a first embodiment which further describes the first aspect, the composition further comprises:
(vi) 1.0% to 3.0% by weight Combination B comprising; xylitylglucoside, anhydroxylitol, and xylitol;
(vii) 0.5% to 2.0% by weight Combination C comprising 70% to 80% panthenol;
(viii) 0.05% to 5% by weight Combination D comprising glycerin, and Globularia alypum leaf extract; and
(ix) 0.25% to 3% by weight niacinamide.

Combination B may assist in emulsion stabilization and gel formation. In further embodiments describing Combination B, Combination B comprises 1% to 25% xylitol, 15% to 35% anhydroxylitol, 35% to 80% xylitylglucoside and water; 3% to 20% xylitol, 20% to 30% anhydroxylitol, 40% to 77% xylitylglucoside and 15% to 17% water; or is Aquaxyl®; and is provided in an amount of 1% to 3% or about 2%. WO 2020/201377 indicates that Aquaxyl® is 3% to 20% xylitol, 20% to 30% anhydroxylitol, 40% to 77% xylitylglucoside and 15% to 17% water.

Combination C is expected to provide for skin conditioning. In further embodiments describing Combination C, Combination C comprises about 75% pantenol; comprises about 75% pantenol, along with about 0.3% citric acid and/or about 1% D-pantolactone, and water; and is provided in an amount of 0.5% to 2.0%, 1% to 1.5%, or about 1.34%.

Combination D is expected to provide skin conditioning and anti-inflammatory activity. In further embodiments describing Combination D, Combination D comprises 85% to 92% glycerin, 4% to 8% Globularia alypum, and 4% to 8% water; about 88% glycerin, about 6% Globularia alypum leaf extract and water (e.g., about 6%) by weight of the composition; or is Phytessence® Blue Daisy. Globularia alypum leaf extract can be obtained using techniques such as those well known in the art, for example, techniques described in U.S. Pat. Nos. 4,263,285 and 9,925,135 (both of which are hereby incorporated by reference herein in their entirety). In different embodiments, Combination D is provided in an amount of 0.05% to 5.0%, or about 0.15% by weight of the composition.

In a second embodiment which further describes the first aspect and the first embodiment, the composition further comprises one or more emollient agents selected from the group consisting of dimethicone, octyldodecanol, undecane, tridecane, dicaprylyl ether, and decyl oleate, wherein the one or more emollient A agents are 2.0% to 11.0% by weight of the composition. In further embodiments, the one or more emollient agents comprise low viscosity dimethicone (in different embodiments providing 4.5-5.5 centistokes (also referred to as cSt) at 25° C. or about 5 centistokes at 25° C.) in an amount providing 1.0% to 4.2%, or about 2.0% by weight of the composition; medium viscosity dimethicone (in different embodiments 332.5-367.5 centistokes at 25° C. or about 350 centistokes at 25° C.) in an amount providing 1.0% to 4.2%, or about 2.0% by weight of the composition; and octyldodecanol in an amount providing 0.5% to 4.2%, or about 1.0% by weight of the composition.

Reference to a particular embodiment includes reference to further embodiments provided therein. For example, reference in the second embodiment to the first embodiment provides a reference to all the embodiments provided in the first embodiment including the further embodiments provided therein.

In a third embodiment which further describes the first aspect, the first embodiment and the second embodiment, the composition further comprises one or more emollients selected from the group consisting of caprylyl glycol, 1,2-hexanediol, and pentylene glycol. In different embodiments, the one or more emollient agents are 0.1% to 2.0%, or about 0.5% by weight of said composition. In further embodiment the one or more emollients are about 50% caprylyl glycol and about 50% 1,2-hexanediol by weight of the composition; or is Symdiol® 68.

In a fourth embodiment which further describes the first aspect, and the first, second and third embodiments, the composition further comprises an absorbent. In further embodiments, the absorbent is either distarch phosphate, Avena sativa starch, or corn modified starch; and the absorbent is provided in a weight percentage of 0.1% to 2.0%, or about 0.5%.

In a fifth embodiment which further describes the first aspect, and the first, second, third and fourth embodiments, the composition further comprises one or more humectants selected from the group consisting of glycerin, sorbitol, and propanediol, wherein the one or more humectants is 4.0% to 15.0% by weight of the composition. In different embodiments, the one or more humectants comprises glycerin in an amount providing 2.0% to 13.0%, or about 6.0% by weight of the composition; and propanediol in an amount providing 2.0% to 13.0%, or about 3.0% by weight of the composition.

In a sixth embodiment which further describes the first aspect, and the first, second, third, fourth and fifth embodiments, the composition further comprises one or more emulsion agents selected from the group consisting of xanthan gum, carbomer, and methylcellulose. In different embodiments, the one or more emulsion agents is provided in an amount of 0.1% to 2.0%, or about 0.5% by weight of the composition. In a further embodiment, the one or more emulsion A agents is xanthan gum.

In a seventh embodiment which further describes the first aspect, and the first, second, third, fourth, fifth and sixth embodiments, the composition further comprises one or more emulsion agents selected from the group consisting of carbomer, sodium acrylate/vinyl alcohol copolymer and sodium polyacryloyldimethyl taurate. In different embodiments, the one or more emulsion agents is provided in an amount of 0.2% to 2.0%, or about 1.0% by weight of the composition. In further embodiments, the one or more emulsion agents is sodium polyacryloyldimethyl taurate; and the sodium polyacryloyldimethyl taurate has a molecule weight of 450 kDa to 550 kDa, or about 500 kDa.

In an eighth embodiment which further describes the first aspect, and the first, second, third, fourth, fifth, seventh and sixth embodiments, the composition further comprises one or more emulsion C agents selected from the group consisting of acrylates/C10-30 alkyl acrylate crosspolymer and carbomer. In different embodiments, the one or more emulsion C agents is 0.1% to 2.0% or about 0.3% by weight of the composition. In further embodiments, the one or more emulsion agents is acrylates/C10-30 alkyl acrylate crosspolymer such as Pemulen® TR-1 and Pemulen® TR-2. Acrylates/C10-30 alkyl acrylate crosspolymers, including Pemulen® TR-1, Pemulen® TR-2 are described, for example, in Tegeli et al., International Journal of Drug Formulation & Research January-February, 2011, Vol. 2 (1), 52-63; and Fiume et al., International Journal of Toxicology, 2017, Vol. 36 (supplement 2) 59S-88S (both of which are hereby incorporated by reference herein in their entirety).

In an ninth embodiment which further describes the first aspect, and the first, second, third, fourth, fifth, sixth, seventh and eighth embodiments, the composition further comprises one or more antioxidants selected from the group consisting of hydroxyacetophenone, pentylene glycol, and caprylyl glycol. In different embodiments, the one or more antioxidants is 0.2% to 2.0%, or about 0.5% by weight of the composition. In a further embodiment, the one or more antioxidants is hydroxyacetophenone.

In a tenth embodiment which further describes the first aspect, and the first, second, third, fourth, fifth, sixth, seventh, eighth and ninth embodiments, the composition further comprises a buffering agent or pH adjuster to provide a pH of 4.0 to 7.0. In further embodiments, the pH is 4 to 6.5, 4 to 7, 4 to 6.5, 4.5 to 6.5 or 4 to 6 and/or the pH adjuster is sodium hydroxide.

In an eleventh embodiment, further describing the first aspect, the composition is as provided in Table 1 for products A, B, C, D, E and F. In different embodiments, each value in Table 1 is independently “about” the indicated value in % by weight.

TABLE 1

| | Product (about weight %) | | | | | |
|---|---|---|---|---|---|---|
| | A | B | C | D | E | F |
| Aqua | 75.68 | 73.68 | 76.77 | 77.02 | 76.77 | 75.77 |
| Glycerin | 6.0 | 7.0 | 5.0 | 6.0 | 5.0 | 6.0 |
| Propanediol | 3.0 | 4.0 | 3.0 | 3.0 | 3.0 | 3.0 |
| Distarch Phosphate | 0.50 | 0.50 | 0.50 | 2.0 | 0 | 0.5 |
| Octyldodecanol | 1.0 | 1.0 | 1.0 | 0.50 | 0.50 | 1.0 |
| Sodium | 1.0 | 1.0 | 1.0 | 1.0 | 1.0 | 1.0 |
| Polyacryloyldimethyl | | | | | | |
| Taurate | | | | | | |
| Caprylyl Glycol & | 0.50 | 0.50 | 0.50 | 0.70 | 1.0 | 0.5 |
| 1,2-Hexanediol | | | | | | |
| Hydroxyacetophenone | 0.50 | 0.50 | 0.50 | 0.50 | 0.50 | 0.5 |
| Acrylates/C10-30 | 0.30 | 0.30 | 0.30 | 0.50 | 0.50 | 0.3 |
| Alkyl Acrylate | | | | | | |
| Crosspolymer (e.g., | | | | | | |
| Pemulen ® TR-1) | | | | | | |
| Xanthan Gum | 0.50 | 0.50 | 0.50 | 0 | 0.30 | 0.5 |
| Sodium Hydroxide | 0.03 | 0.03 | 0.03 | 0.1 | 0.5 | 0.03 |
| Dimethicone, medium | 2.0 | 2.0 | 2.0 | 0.03 | 0.03 | 2.0 |
| viscosity, preferably | | | | | | |
| about 350 cSt (e.g., | | | | | | |
| Xiameter ™ PMX-200 | | | | | | |
| silicone fluid 350 cSt) | | | | | | |
| Polyglutamic Acid | 0.10 | 0.10 | 0.10 | 1.0 | 2.0 | 0.10 |
| Combination A | 2.0 | 2.0 | 2.0 | 0.50 | 0.10 | 2.0 |
| Allantoin | 0.30 | 0.30 | 0.30 | 2.0 | 2.0 | 0.30 |
| Hydrolyzed | 0.10 | 0.10 | 0.01 | 0.50 | 0.30 | 0.01 |
| Hyaluronic Acid | | | | | | |
| Combination D | 0.15 | 0.15 | 0.15 | 0.40 | 0 | 0.15 |
| Combination C | 1.34 | 1.34 | 1.34 | 0.10 | 0.01 | 1.34 |
| Combination B | 2.0 | 2.0 | 2.0 | 0.15 | 0.15 | 2.0 |
| Dimethicone low | 2.0 | 2.0 | 2.0 | 1.0 | 1.34 | 2.0 |
| viscosity, preferably | | | | | | |
| about 5 cSt (e.g., | | | | | | |
| Xiameter ™ PMX-200 | | | | | | |
| silicone fluid 5 cSt ) | | | | | | |
| Niacinamide | 1.0 | 1.0 | 1.0 | 2.0 | 2.0 | 1.0 |

Wherein in Table 1:
Combination A in different embodiments is (1) PatcH2O®; or (2) comprises by weight percent: about 17.5% glycerin; about 1000 trehalose; about 1000 urea; about 3% seine; about 2% pentylene glycol; about 0.50% algin or a cosmetically acceptably salt thereof, having a molecular weight of 100 to 550 kDa, preferably sodium alginate; about 0.30% glyceryl polyacrylate; about 0.50% caprylyl glycol; about 0.250% hyaluronic acid or a cosmetically acceptable salt thereof, having a molecular weight of 250 to 450 kDa, preferably sodium hyaluronate; about 0.25% pullulan, having a molecular weight of 100 to 250 kDa; and about 55.500 water; in a further embodiment combination A further comprises about 0.10% disodium phosphate and about 0.020% potassium phosphate;
Combination B in different embodiments comprises by weight percent 1% to 25% xylitol, 15% to 35% anhydroxylitol, 35% to 80% xylitylglucoside; 3% to 20% xylitol, 20% to 30% anhydroxylitol, 40% to 77% xylitylglucoside and 15% to 17% water; or is Aquaxyl®;
Combination C in different embodiments comprises by weight percent about 75% pantenol; or comprises about 75% pantenol, about 0.3% citric acid and/or about 1% D-pantolactone, and water;
Combination D in different embodiments comprises by weight percent about 88% glycerin, about 6% Globularia alypum leaf extract and about 6% water; or is Phytessence® Blue Daisy; and
Caprylyl glycol and 1,2-hexanediol in different embodiments are provided in an amount by weight percent about 50% caprylyl glycol and about 50%; or is Symdiol® 68.

In a twelfth embodiment which further describes the first aspect, and the first, second, third, fourth, fifth, sixth, seventh, eighth, ninth, tenth and eleventh embodiments, the composition is an oil free gel.

In a thirteenth embodiment which further describes the first aspect, and the first, second, third, fourth, fifth, sixth, seventh, eighth, ninth, tenth, eleventh and twelfth embodiments, the composition is a semi-transparent, water gel.

### II. SKIN TREATMENT

Proper skin water content is important for skin health and appearance. The compositions described herein can be used to provide for skin hydration and moisturization, treating dry skin, and/or treating sensitive skin. Skin hydration agents are able to attract water, while moisturization agents prevent water loss.

Dehydrated skin is due to a lack of proper water content and can cause one or more of the following: skin tightness, irritation, redness, sensitivity, roughness, dullness, oiliness and breakouts. Dehydrated skin can be treated, for example, using agents providing for rehydration.

In certain embodiments, a composition provided herein enhances skin hydration (water content). The level of skin hydration can be measured using techniques such as a Corneometer® described in the examples below. An effective amount of a composition used for skin hydration is that amount providing a detectable increase in skin hydration.

In certain embodiments, skin hydration is maintained, or preferably, enhanced compared to just prior to the initial treatment, by applying the composition one or more times in a 24 hour period, one time about every 48 hours, or one time about every 72 hours. In further embodiments, the amount is sufficient to enhance skin hydration compared to the initial hydration level for at least about 48 hours and/or up to about 72 hours.

In certain embodiments, a composition provided herein moisturizes (inhibits water loss). The ability of a composition to reduce water loss can be measured using techniques such as Trans-epidermal Water Loss (TEWL) described in the examples below. An effective amount of a composition used for skin moisturization is that amount resulting in a detectable decrease in water loss, compared to untreated skin.

In certain embodiments, water loss is inhibited, compared to just prior to the initial treatment, by applying the composition one or more times in a 24 hour period, one time about every 48 hours, and one time about every 72 hours. In further embodiments, the amount is sufficient to inhibit water loss, compared to just prior to the initial treatment, for at least about 48 hours and/or up to about 72 hours.

In certain embodiments, compositions provided here are used to treat sensitive skin. Sensitive skin can be clinically defined by characteristic abnormal sensory perceptions including tightness, abnormal stinging, burning, tingling and pain. In many cases, symptoms are transient and unaccompanied by visual dermatologically responses. An effective amount of a composition for treating sensitive skin, is an amount that reduces one or more symptoms of sensitive skin.

While the determination of the abnormal sensations is subjective, diagnosis of sensitive skin can be assisted using a variety of different tests such as sensory testing methods. Examples of such methods include stinging tests with lactic acid, or capsaicin and dimethylsulfoxide; occlusion tests; behind the knees test; washing and exaggerated immersion tests, and quantitative sensory testing (QST). (Misery et al., JEADY 2016, 30 (Suppl. 1), 2-8, hereby incorporated by reference herein in its entirety).

In an embodiment, sensitive skin is measured using a Sensiscale. The Sensiscale measures skin sensitivity through a questionnaire evaluating subjective skin irritability, stinging, burning, sensations of heat, tautness, itching, pain, general discomfort, flushes (hot flashes); and redness appearance. A score greater than 20 indicates sensitive skin. (Misery et al., Acta Derm Venereol 2014, 94: 635-639, hereby incorporated by reference herein in its entirety).

In certain embodiments, sensitive skin is treated by applying the composition one or more times in a 24 hour period, one time about every 48 hours, and one time about every 72 hours. In further embodiments, the amount is sufficient to treat sensitive skin for at least about 48 hours and/or is sufficient to treat sensitive skin for up to about 72 hours.

In certain embodiments, the compositions provided here are used to treat dry skin. Dry skin can occur when the skin loses water and oil. Symptoms of dry skin include skin dehydration, flakes, cracks, pruritus, and peeling. An effective amount of a composition for treating dry skin, is an amount that reduces one or more symptoms of dry skin. In certain embodiments, dry skin is treated by applying the composition one or more times in a 24 hour period, one time about every 48 hours, and one time about every 72 hours. In further embodiments, the amount is sufficient to treat dry skin for at least about 48 hours and/or is sufficient to treat dry skin for up to about 72 hours.

In certain embodiments, the composition is used as a facial mask for treating dry skin comprising the step of applying an effective amount of the composition as a mask on the face 2 to 3 times a week for 10 to 20 minutes. In further embodiments, the composition is applied as a mask on the face 2 to 3 times a week for 10 to 15 minutes, or about 15 minutes.

### EXAMPLES

Examples are provided below further illustrating different features of the present invention and methodology for practicing the invention. The provided examples do not limit the claimed invention.

### Example 1: Product A Production

The present example illustrates methods that may be used to produce Product A. Different components and steps where the components were utilized in producing Product A are provided in Table 2. The particular amounts of components utilized can be chosen based on the desired scale. For example, to obtain 100 g of the composition, the % in Table 2 will correspond to grams.

TABLE 2

| | | | Weight % in the |
|---|---|---|---|
| Step | Commercial Name | INCI Name | formula |
| 1 | Water, Purified | Aqua | 75.68% |
| 1 | Glycerine | Glycerin | 4.00% |
| 1 | 1,3-Propanediol | Propanediol | 3.0% |
| 1 | Allantoin_OTC | Allantoin | 0.30% |
| 1 | Pemulen ™ TR-1 | Acrylates/C10-30 Alkyl Acrylate | 0.30% |
| | Polymer | Crosspolymer | |
| 2 | Glycerine | Glycerin | 1.00% |
| 2 | SymDiol ® 68 | Caprylyl Glycol 1,2-Hexanediol | 0.50% |
| 2 | SymSave ® H | Hydroxyacetophenone | 0.50% |
| 3 | Aquaxyl ™ | Xylitylglucoside | 2.00% |
| | | Anhydroxylitol | |
| | | Aqua | |
| | | Xylitol | |
| 3 | D-Panthenol 75 L | Panthenol | 1.34% |
| | | Aqua | |
| | | Pantolactone | |
| | | Citric Acid | |
| 3 | Phytessence ™ Blue | Glycerin | 0.15% |
| | Daisy | Globularia Alypum Leaf Extract | |
| | | Aqua | |
| 3 | PatcH2O ® | Aqua, Glyercin, Trehalose, Urea, | 2.00% |
| | | Serine, Pentylene Glycol, Algin, | |
| | | Glyceryl Polyacrylate, Caprylyl Glycol, | |
| | | Sodium Hyaluronate, Pullulan, | |
| | | Disodium Phosphate, Potassium | |
| | | Phosphate | |
| 3 | Hydrolyzed | Hydrolyzed Hyaluronic Acid | 0.10% |
| | Hyaluronic Acid | | |
| | (HA-TLM 3-5) | | |
| 3 | Hyafactor ™-PGA | Polyglutamic Acid | 0.10% |
| 3 | Niacinamide USP | Niacinamide | 1.00% |
| 4 | Xantural 75 | Xanthan Gum | 0.50% |
| 4 | Glycerine | Gylcerin | 1.00% |
| 5 | Aristoflex ® Silk | Sodium Polyacryloylidmethyl Taurate | 1.00% |
| 5 | Corn PO4 PH “B” | Distarch Phosphate | 0.50% |
| 6 | XIAMETER ™ | Dimethicone | 2.00% |
| | PMX-200 Silicon | | |
| | Fluid 5 cSt | | |
| 6 | XIAMETER ™ | Dimethicone | 2.00% |
| | PMX-200 Silicone | | |
| | Fluid 350 cSt | | |
| 6 | Eutanol ® G | Octyldodecanol | 1.00% |
| 7 | Sodium Hydroxide | Sodium Hydroxide | 0.03% |
| | Pellets NF | | |

Step 1: Aqua, glycerin, and 1,3-propanediol were combined in a suitably sized stainless-steel reactor (“main container”), stirred, and heated at 50° C.±5° C. until dissolved. Then allantoin was added to the reactor. The solution was stirred until complete dispersion of allantoin, as indicated by the absence of white dots. The solution was cooled at 25° C.±5° C. Pemulen™ TR-1 polymer was then added to the solution and the solution was stirred until complete dispersion.
Step 2: Glycerin, SymDiol® 68 and SymSave® H were combined in a clean container and stirred. The resulting mixture was added to the main container from Step 1.
Step 3: Aquaxyl™, D-Panthenol 75 L, Phytessence™ Blue Daisy, PatcH₂O® Hydrolyzed hyaluronic acid (HA-TLM 3-5), Hyafactor™-PGA, and niacinamide were added one by one, while stirring, to the main container from step 2. The mixture was stirred until complete dispersion of all the components.
Step 4: Glycerin and Xantural 75 were combined in a clean container, stirred, and added to the solution from Step 3.
Step 5: Aristoflex® Silk was added to the main container from Step 4 and stirred until complete incorporation, as determined by the absence of lumps. Corn PO4 PH “B” was then added and the solution was stirred until complete incorporation, as determined by the absence of lumps.
Step 6: XIAMETER™ PMX-200 Silicon Fluid 5 CST, XIAMETER™ PMX-200 Silicone Fluid 350 cSt, and Eutanol® G were added, one by one, under stirring, to the main container from Step 5. The ingredients were vigorously mixed until complete incorporation. The resulting solution formed a semi-transparent white gel.
Step 7: The pH of the solution was adjusted, if needed, to a pH of 4.5-5.5 using sodium hydroxide.

### Example 2: Effect of Product A on Sensitive Skin

A clinical study examining the efficacy of Product A (produced as described in Example 1) on subjects with sensitive skin was performed. The study was made up of the following groups:
1. Group “Classic”: Subjects applied Product A on their face under technical supervision.
2. Group “Mask”: The subjects tested Product A two to three times a week whenever needed on the face at home. The subjects applied a generous layer of the gel on their clean skin and left the product on for 15 minutes before wiping it off and gently massaging the remaining product into their skin.
3. Group “Classic”+“Mask”: Made up of the “Mask” group and a subset (20 subjects) of the “Classic” group. The subjects tested Product A on their face and forearms twice a day (morning and evening) at home under normal conditions of use as a cream.

Study duration was 28 days, with subjects evaluated on Days 0, 7, and 28. The evaluations included the subjects completing a questionnaire to obtain a Sensiscale score (classic group), evaluation by dermatologist, and completing a subjective questionnaire about skin sensitivity (classic and mask groups). During Day 0, the classic group applied product prior to the subjective evaluation. The Sensiscale is a 10-point scale, based on a subject evaluation of different skin condition, with higher scores corresponding to increased skin sensitivity. The dermatologist preformed a clinical examination (classic and mask groups) for signs reported by subjects (functional and physical signs); and graded the cutaneous state of dryness and roughness (visual evaluation), softness and suppleness (tactical evaluations). The subjective questionnaire is a self-evaluation on the effect of the product on the skin. The classic group filled in the subjective questionnaire on site, and the mask group filled in a subjective questionnaire at home after first use of the product.

The “Classic” group comprised 43 subjects, while the “Mask” group comprised 20 subjects (63 subjects total). The average age of the subjects was 39 years (SEM of ±2 years) with a minimum age 19 and the maximum age 60. All subjects were female, had a skin phototype of I to IV, and had sensitive skin (determined by questionnaire) and dehydrated skin (declarative under dermatologic control).

Both mask and classic application of Product A was well-tolerated on sensitive skin. The majority of subjects in the “Classic” group (96%) and in the “Mask” group (75%) exhibited no abnormal clinical signs such as pustules, erythema, or desquamation that worsened compared to Day 0 of the study. Additionally, the majority of the subjects in the “Classic” group (75%) and in the “Mask” group (80%) did not exhibit functional and physical signs of skin aversion such as tightness, stinging, itching, warm burning sensation, erythema, edema, dryness, desquamation, and roughness.

In the “Classic” group, 11 subjects reported function and/or physical signs (one of which was retained as relevant) and 2 subjects presented clinical signs (one of which was retained as relevant). In the “Mask” group, 4 subjects reported functional and/or physical signs (two of which was retained as relevant); and 5 subjects presented clinical signs, which were not retained as relevant.

The results of treatment with Product A on suppleness score, softness score, and roughness score were scored for the Classic group. Topical application of Product A resulted in a significant increase in suppleness score: +16% (p=0.0202) after 7 days of treatment and +30% (p=0.0005) after 28 days of treatment (Table 3). These effects were observed in 48% and 73% of the subjects, respectively. Treatment with Product A significantly increased the softness score in 58% of the subjects: +15% (p=0.0308) after 28 days (Table 3). Moreover, treatment with Product A significantly increased the roughness score in 73% of the subjects: +19% (p=0.0005) after 28 days (Table 3). Consequently, Product A improved the skin's state and aspect.

TABLE 3

| | | | Roughness/Skin |
|---|---|---|---|
| | Suppleness: | Softness: | Texture: |
| Day | Average Grade | Average Grade | Average Grade |
| 0 | 5.6 | 6.6 | 6.0 |
| 7 | 6.6 | 6.6 | 5.9 |
| 28 | 7.3 | 7.8 | 7.2 |

In addition to the skin's state and aspect, treatment with Product A induced a significant decrease in the dryness score: −23% (p=0.0062) after 7 days and −52% (p<0.00001) after 28 days (Table 4). These effects were observed in 56% and 96% of the subjects, respectively. Therefore, Product A induced a moisturizing effect.

TABLE 4

| Day | Dryness: Average Grade |
|---|---|
| 0 | 5.8 |
| 7 | 4.5 |
| 28 | 2.7 |

Topical treatment with Product A also decreased skin sensitivity. Treatment with Product A induced a significant decrease in skin sensitivity (for the global score) by −56% (p<0.0001) after 7 days and −70% (p<0.0001) after 28 days (Table 5). These effects were observed in 95% and 94% of the subjects, respectively. Moreover, each individual score significantly decreased on Day 7 and Day 28.

TABLE 5

| Day | Sensiscale (A.U.) |
|---|---|
| 0 | 40.9 |
| 7 | 17.8 |
| 28 | 12.0 |

The majority of subjects for the “Mask” group and the “Classic” group noticed an improvement in skin sensitivity. There were 30 subjects in the “Classic” group who completed the questionnaire about skin sensitivity and 20 subjects in the “Mask” group completed the questionnaire.

### Example 3: Effect of Product A on Dry Skin

A clinical study examining the moisturizing and protective effect of Product A, produced as described in Example 1, was conducted in subjects with dry skin. The study comprised two subject groups who were tested on a defined treatment zone on their forearms at an investigation center.

The subjects arrived at the investigation center without having applied any product to their forearms since the previous evening and were allowed to acclimatize for about 30 minutes in an air-conditioned waiting room with their forearms bared each time they arrived at the study investigation center. A technician defined three zones on the subject's forearms for Corneometer® measurements: two treatment zones and one non-treated zone (the control). Transepidermal Water Loss (TEWL) following treatment with Product A was measured using a Tewameter® TM 300 probe. Cutaneous hydration rates were measured using Corneometer® CM 825 on the three defined zones. Initial measurements for TEWL and the cutaneous hydration rate on two zones (a treated and a untreated zone) were taken at a defined time 0 (t0). All subjects were provided with daily logs to complete over the course of study that questioned the subjects about potential intolerance sensations they felt or observed. The product was lightly and uniformly applied onto the subjects' skin with a fingerstall. Subjects returning to the investigation center for TEWL and Corneometer® measurements, were instructed not wash their forearms after the initial application.

Measurements were taken following treatment with Product A at 15 minutes, 2 hours, 4 hours, 8 hours, 24 hours, 48 hours, and 72 hours. Cutaneous hydration rates were measured at each time point, while TWEL measurements were taken up until 24 hours following product application. When both Tewameter® measurements and Corneometer® measurements were collected, Tewameter® measurements were performed first. Before each Corneometer® measurement each zone was wiped three times with tissue paper. Possible adverse reactions were recorded at 24 hours, 48 hours, and 72 hours following treatment.

The treatment group was made up to 22 female subjects, average age of 36 years (SEM of ±3 years) with a minimum age of 22 and maximum age of 61. All of the subjects had dry to very dry skin on the forearms (cutaneous hydration rate 35-50 A.U. for dry skin and <35 A.U. for very dry skin, verified using Corneometer®), and had a skin phototype of I to IV.

Subjects treated with a single application of Product A exhibited a cutaneous barrier maintenance until 24 hours as indicated by TEWL measurements. At 24 hours post-treatment, 90% of subjects exhibited a cutaneous barrier maintenance with −11% of TEWL (P<0.001) compared to a non-treated zone. A summary of the TEWL measurements for subjects treated with Product A is shown in Table 6.

TABLE 6
No adverse events were observed during the study.

| Time (h = hours) | TEWL Loss |
|---|---|
| Δt2 h | −9%* |
| Δt4 h | −11%* |
| Δt8 h | −14%* |
| Δt24 h | −11%* |

*Significant values

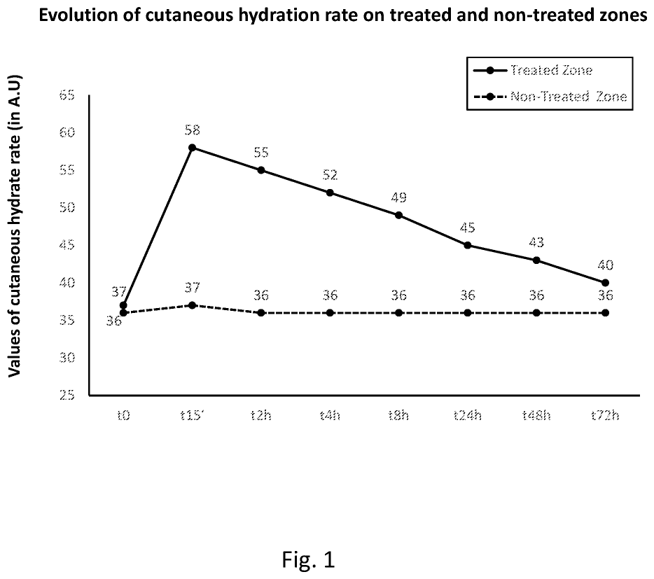

Subjects treated with a single application of Product A exhibited a statistically significant long-lasting moisturizing effect (FIG. 1). At 72 hours, subjects exhibited a cutaneous hydration rate of +8% compared to the non-treated zone (Table 7).

TABLE 7

| | |
|---|---|
| | % Variations of Cutaneous |
| Time | Hydration Rate |

| | | |
|---|---|---|
| 15 | minutes | +57% |
| 2 | hours | +49% |
| 4 | hours | +41% |
| 8 | hours | +33% |
| 24 | hours | +22% |
| 48 | hours | +15% |
| 72 | hours | +8% |

### Example 4: Facial Application of Product A

The effect of Product A, produced as described in Example 1, on fine lines, skin dryness, roughness, radiance, and redness was examined in subjects with sensitive or dehydrated skin. The study comprised one subject group termed the “Classic” group, who used Product A on their face under normal conditions, twice-daily applications in the morning and evening.

On Day 0, the subjects arrived at the investigation center without having applied product to their face in the morning (except morning wash with water only 4 hours before the visit) and were allowed to acclimatize for 15 minutes. A technician defined measurement zones on the cheek and underneath the eye and obtained acquisitions in 2D (on the cheek) and 3D (on underneath the eye) using C-Cube®. The C-Cube 2® dermoscope allows skin image acquisition in Ultra High Definition. The subjects applied the product in the investigation center under technician control. Additionally, the subjects were provided with a daily log (Day 0 to Day 7) to record possible unpleasant signs or medications and the frequency of product applications.

On Day 7 (last application performed on the previous day), the subjects returned to the investigation center and the technician defined the same measurement zones as Day 0. Measurements were again obtained in 2D and 3D using C-Cube®. The subjects were provided with a daily log to record possible unpleasant signs or medications and the frequency of product applications for Day 7 to Day 28.

On Day 28 (last application performed on the previous day), the subjects returned to the investigation center and a technician obtained 2D and 3D measurements in the same zones using C-Cube®.

The “Classic” group comprised 43 subjects. The average age of the subjects was 41 years (SEM of ±2 years) with a minimum age of 19 and the maximum age of 60. All of the subjects were female, had sensitive skin (verified by sensitive skin questionnaire) or dehydrated skin (declarative under dermatologic control), and a skin phototype of I to IV.

All results were obtained in subjects who had a skin dryness score greater than 3 on Day 0. Treatment with Product A induced a significant decrease in the Sdr parameter: −21% (p=0.0225) after 28 days of use in 77% of the subjects and the Elevation parameter tended to 0 on Day 7 and Day 28 in 62% of the subjects. The Sdr parameter is the ratio of the developed interfacial area, expressed as the percentage of the additional surface of the definition area provided by the texture compared to the flat definition area. Elevation is the actual height of the points in the ROI (region of interest) relative to the mean elevation, expressed in μm. The results demonstrate Product A induced an improvement in skin texture.

Treatment with Product A did not induce any significant variations of a* parameter and Erythema index immediately after its application, and after 7 and 28 days of use. A slight increase in L* parameter was observed on Day 7 and Day 28 (respectively +1% and +2%), which reflected lighter skin. The L* parameter (luminance) reflects the dark character of a redness. Software converts image pixels into colorimetric parameters from the standard CIE L*a*b*. The a* parameter is a chrominance parameter for the green-to-red spectrum.

Furthermore, application of Product A resulted in more radiant skin (Table 8). There was a significant increase in radiance index (+6%, p=0.0072) on Day 28 in 77% of the subjects. Consequently, Product A improved skin radiance after 28 days of twice-daily use.

TABLE 8

| Day | Radiance Index (A.U.) |
|---|---|
| 0 | 58.4 |
| 0Timm | 57.3 |
| 7 | 59.7 |
| 28 | 62.1 |

Timm refer to Time Immediate; up to 15 minutes maximum after product is applied.

In addition to improving skin radiance and skin texture, use of Product A moisturized the skin. Application of Product A resulted in a significant decrease in the desquamation index (Table 9). On Day 0Timm there was a 6% decrease in the desquamation index in 47% of the subjects. On Day 7 there was a 4% decrease in the desquamation index in 39% of the subjects. Use of Product A led to a significant decrease (36%, p=0.000) in the desquamation index in 77% of the subjects on Day 28. Thus, Product A resulted in a moisturizing effect after 28 days of twice-daily use.

TABLE 9

| Day | Desquamation Index (%) |
|---|---|
| 0 | 1.12 |
| 0Timm | 1.05 |
| 7 | 1.08 |
| 28 | 0.67 |

### Example 5: Clinical Study Evaluating the Moisturizing Effect of 5 Different Cosmetic Products After a Single Cutaneous Application

A clinical study was conducted to examine the moisturizing effect of 5 different cosmetic products by repeated instrumental measurements of the skin electrical capacitance (corneometry), after a single cutaneous application in adult female subjects. The cosmetic products were tested on the left/right forearm (inner side) and compared to a control area on the forearm in each subject.

The 5 cosmetic products tested are depicted in Table 10.

TABLE 10

| | | Formula | Formula | Formula | Formula | Formula |
|---|---|---|---|---|---|---|
| Raw Material | INCI Name | 1 | 2 | 3 | 4 | 5 |
| Water, Purified | Aqua | 88.67 | 78.17 | 76.17 | 75.87 | 75.77 |
| 1,3-Propanediol | Propanediol | 3.00 | 3.00 | 3.00 | 3.00 | 3.00 |
| XIAMETER ™ | Dimethicone | 2.00 | 2.00 | 2.00 | 2.00 | 2.00 |
| PMX- 200 Silicone | | | | | | |
| Fluid 350 CS | | | | | | |
| XIAMETER ™ | Dimethicone | 2.00 | 2.00 | 2.00 | 2.00 | 2.00 |
| PMX- 200 Silicone | | | | | | |
| 5 CST OTC | | | | | | |
| Aristoflex ® Silk | Sodium | 1.00 | 1.00 | 1.00 | 1.00 | 1.00 |
| | Polyacryloylidmethyl | | | | | |
| | Taurate | | | | | |
| Eutanol ® G | Octyldodecanol | 1.00 | 1.00 | 1.00 | 1.00 | 1.00 |
| Symdiol ® 68 | Caprylyl Glycol & 1, 2- | 0.50 | 0.50 | 0.50 | 0.50 | 0.50 |
| | Hexanediol | | | | | |
| SymSave ® H | Hydroxyacetophenone | 0.50 | 0.50 | 0.50 | 0.50 | 0.50 |
| Corn PO4 PH “B” | Distarch Phosphate | 0.50 | 0.50 | 0.50 | 0.50 | 0.50 |
| Xantural 75 | Xanthan Gum | 0.50 | 0.50 | 0.50 | 0.50 | 0.50 |
| Pemulen ™ TR-1 | Acrylates/C10-30 Alkyl | 0.30 | 0.30 | 0.30 | 0.30 | 0.30 |
| Polymer | Acrylate Crosspolymer | | | | | |
| Sodium Hydroxide | Sodium Hydroxide | 0.03 | 0.03 | 0.03 | 0.03 | 0.03 |
| Pellets NF | | | | | | |
| Hyafactor ™-PGA | Polyglutamic Acid | 0.00 | 0.00 | 0.00 | 0.00 | 0.10 |
| Niacinamide USP | Niacinamide | 0.00 | 1.00 | 1.00 | 1.00 | 1.00 |
| Phytessence ™ | Glycerin & | 0.00 | 0.15 | 0.15 | 0.15 | 0.15 |
| Blue Daisy | Globularia Alypum Leaf | | | | | |
| | Extract & Aqua | | | | | |
| D-Panthenol 75 L | Panthenol& Aqua & | 0.00 | 1.34 | 1.34 | 1.34 | 1.34 |
| | Pantolactone & | | | | | |
| | Citric Acid | | | | | |
| Aquaxyl ™ | Xylitylglucoside & | 0.00 | 2.00 | 2.00 | 2.00 | 2.00 |
| | Anhydroxylitol & | | | | | |
| | Aqua & Xylitol | | | | | |
| Allantoin OTC | Allantoin | 0.00 | 0.00 | 0.00 | 0.30 | 0.30 |
| Glycerine | Glycerine | 0.00 | 6.00 | 6.00 | 6.00 | 6.00 |
| Hydrolyzed | Hydrolyzed Hyaluronic | 0.00 | 0.01 | 0.01 | 0.01 | 0.01 |
| Hyaluronic Acid | Acid | | | | | |
| (HA-TLM-35) | | | | | | |
| PatcH2O ® | Aqua & Glyercin & | 0.00 | 0.00 | 2.00 | 2.00 | 2.00 |
| | Trehalose & Urea & Serine | | | | | |
| | & Pentylene Glycol& Algin | | | | | |
| | & Glyceryl Polyacrylate & | | | | | |
| | Caprylyl Glycol & Sodium | | | | | |
| | Hyaluronate & Pullulan & | | | | | |
| | Disodium Phosphate & | | | | | |
| | Potassium Phosphate | | | | | |

The study comprised 22 female subjects who ranged in age from 18-70 years old. All of the subjects were considered healthy and did not have a skin disease such as eczema, lichen or planus; or other disorder such as cardiovascular disorder or endocrine disorder. Out of the 22 subjects enrolled in the study, 21 subjects came to the investigation center, and 20 were included in the study (one subject did not return for certain measurements). The analysis of the results was performed on 19 subjects ranging in age from 25 to 70 years old (mean age of 56) who presented with dry skin on the forearm (inner side) (electric capacitance ≤50 a.u.).

The subjects were treated with a single application of the cosmetic product of interest to a treatment area on their forearms (determined according to randomization) by a technician. Measurements were performed with a Comeometer™ on the treated areas and control area at TO (baseline), 2 hours (T2), 4 hours (T4), 8 hours (T8), 24 hours (T24), and 72 hours (T72) following application. Electrical capacitance (a.u.) following treatment with Formula 1 is shown in Table 11 and a summary of the moisturizing effect in response to treatment with formula 1 is shown in Table 12.

TABLE 11

| | | | | | Probability | |
|---|---|---|---|---|---|---|
| | | | | | p: Student | |
| | Control area | | Treated area | | “t” test or | |
| | | Standard | | Standard | Wilcoxon | |
| | Mean | deviation | Mean | deviation | test | Conclusion |
| T0 | 32.28 | 6.06 | 31.48 | 4.14 | 0.495 | N.S. |
| | | | | | (Student) | |
| T2 hours-T0 | 0.13 | 1.31 | 2.02 | 3.18 | 0.016 | S. |
| | | | | | (Student) | |
| T4 hours-T0 | 0.21 | 2.05 | 1.65 | 2.91 | 0.066 | L.S. |
| | | | | | (Student) | |
| T8 hours-T0 | 0.01 | 1.89 | 1.39 | 2.75 | 0.067 | L.S. |
| | | | | | (Student) | |
| T24 hours-T0 | 0.35 | 2.49 | −0.36 | 2.71 | 0.277 | N.S. |
| | | | | | (Student) | |
| T48 hours-T0 | 0.33 | 2.62 | −1.19 | 3.67 | 0.010 | S. (NF) |
| | | | | | (Wilcoxon) | |
| T72 hours-T0 | 0.35 | 2.43 | −0.34 | 3.04 | 0.267 | N.S. |
| | | | | | (Student) | |

Note:
N = 19; S. = Statistically significant probability (p < 0.05); L.S. = Probability close to
significant (0.05 ≤ p < 0.10); N.S. = Not significant probability (p ≥ 0.10)

TABLE 12

| Time point evaluation | | Moisturization gain | Statistical Significance |
|---|---|---|---|
| T2 | hours | +6.01% | Yes |
| T4 | hours | | No |
| T8 | hours | | No |
| T24 | hours | | No |
| T48 | hours | | Yes, but favorable |
| T72 | hours | | No |

Electrical capacitance (a.u.) following treatment with Formula 2 is shown in Table 13 and a summary of the moisturizing effect in response to treatment with Formula 2 is shown in Table 14.

TABLE 13

| | Control area | | Treated area | | Probability | |
|---|---|---|---|---|---|---|
| | | Standard | | Standard | p: Student | |
| | Mean | deviation | Mean | deviation | “t” test | Conclusion |
| T0 | 32.28 | 6.06 | 30.16 | 4.95 | 0.044 | S. |
| T2 hours-T0 | 0.13 | 1.31 | 11.88 | 5.42 | <0.001 | S. |
| T4 hours-T0 | 0.21 | 2.05 | 11.89 | 5.27 | <0.001 | S. |
| T8 hours-T0 | 0.01 | 1.89 | 11.77 | 4.90 | <0.001 | S. |
| T24 hours-T0 | 0.35 | 2.49 | 4.15 | 2.74 | <0.001 | S. |
| T48 hours-T0 | 0.33 | 2.62 | 2.34 | 3.61 | 0.038 | S. |
| T72 hours-T0 | 0.35 | 2.43 | 2.33 | 2.74 | 0.016 | S. |

Note:
N = 19; S. = Statistically significant probability (p < 0.05); L.S. = Probability close to significant (0.05 ≤ p < 0.10); N.S. = Not significant probability (p ≥ 0.10)

TABLE 14

| Time point evaluation | | Moisturization gain | Statistical Significance |
|---|---|---|---|
| T2 | hours | +38.99% | Yes |
| T4 | hours | +38.80% | Yes |
| T8 | hours | +38.99% | Yes |
| T24 | hours | +12.68% | Yes |
| T48 | hours | +6.70% | Yes |
| T72 | hours | +6.31% | Yes |

Electrical capacitance (a.u.) following treatment with Formula 3 is shown in Table 15 and a summary of the moisturizing effect in response to treatment with Formula 3 is shown in Table 16.

TABLE 15

| | Control area | | Treated area | | Probability | |
|---|---|---|---|---|---|---|
| | | Standard | | Standard | p: Student | |
| | Mean | deviation | Mean | deviation | “t” test | Conclusion |
| T0 | 32.28 | 6.06 | 30.12 | 4.64 | 0.064 | L.S. |
| T2 hours-T0 | 0.13 | 1.31 | 14.58 | 4.11 | <0.001 | S. |
| T4 hours-T0 | 0.21 | 2.05 | 13.65 | 3.77 | <0.001 | S. |
| T8 hours-T0 | 0.01 | 1.89 | 12.93 | 5.86 | <0.001 | S. |
| T24 hours-T0 | 0.35 | 2.49 | 5.21 | 3.43 | <0.001 | S. |
| T48 hours-T0 | 0.33 | 2.62 | 2.75 | 3.62 | 0.010 | S. |
| T72 hours-T0 | 0.35 | 2.43 | 2.41 | 3.15 | 0.009 | S. |

Note:
N = 19; S. = Statistically significant probability (p < 0.05); L.S. = Probability close to significant (0.05 ≤ p < 0.10); N.S. = Not significant probability (p ≥ 0.10)

TABLE 16

| Time point evaluation | | Moisturization gain | Statistical Significance |
|---|---|---|---|
| T2 | hours | +48.00% | Yes |
| T4 | hours | +44.70% | Yes |
| T8 | hours | +42.90% | Yes |
| T24 | hours | +16.21% | Yes |
| T48 | hours | +8.11% | Yes |
| T72 | hours | +6.92% | Yes |

Electrical capacitance (a.u.) following treatment with Formula 4 is shown in Table 17 and a summary of the moisturizing effect in response to treatment with Formula 4 is shown in Table 18.

TABLE 17

| | Control area | | Treated area | | Probability | |
|---|---|---|---|---|---|---|
| | | Standard | | Standard | p: Student | |
| | Mean | deviation | Mean | deviation | “t” test | Conclusion |
| T0 | 32.28 | 6.06 | 32.07 | 5.87 | 0.846 | N.S. |
| T2 hours-T0 | 0.13 | 1.31 | 10.16 | 4.42 | <0.001 | S. |
| T4 hours-T0 | 0.21 | 2.05 | 11.74 | 4.70 | <0.001 | S. |
| T8 hours-T0 | 0.01 | 1.89 | 13.38 | 4.76 | <0.001 | S. |
| T24 hours-T0 | 0.35 | 2.49 | 4.16 | 3.98 | 0.001 | S. |
| T48 hours-T0 | 0.33 | 2.62 | 1.62 | 4.62 | 0.252 | N.S. |
| T72 hours-T0 | 0.35 | 2.43 | 1.09 | 3.09 | 0.203 | N.S. |

Note:
N = 19; S. = Statistically significant probability (p < 0.05); L.S. = Probability close to significant (0.05 ≤ p < 0.10); N.S. = Not significant probability (p ≥ 0.10)

TABLE 18

| Time point evaluation | | Moisturization gain | Statistical Significance |
|---|---|---|---|
| T2 | hours | +31.28% | Yes |
| T4 | hours | +35.99% | Yes |
| T8 | hours | +41.69% | Yes |
| T24 | hours | +11.89% | Yes |
| T48 | hours | — | No |
| T72 | hours | — | No |

Electrical capacitance (a.u.) following treatment with Formula 5 is shown in Table 19 and a summary of the moisturizing effect in response to treatment with Formula 5 is shown in Table 20.

TABLE 19

| | Control area | | Treated area | | Probability | |
|---|---|---|---|---|---|---|
| | | Standard | | Standard | p: Student | |
| | Mean | deviation | Mean | deviation | “t” test | Conclusion |
| T0 | 32.28 | 6.06 | 29.65 | 4.35 | 0.014 | S. |
| T2 hours-T0 | 0.13 | 1.31 | 12.73 | 5.36 | <0.001 | S. |
| T4 hours-T0 | 0.21 | 2.05 | 13.84 | 4.45 | <0.001 | S. |
| T8 hours-T0 | 0.01 | 1.89 | 14.28 | 4.86 | <0.001 | S. |
| T24 hours-T0 | 0.35 | 2.49 | 6.56 | 3.48 | <0.001 | S. |
| T48 hours-T0 | 0.33 | 2.62 | 4.09 | 3.41 | 0.001 | S. |
| T72 hours-T0 | 0.35 | 2.43 | 4.18 | 3.50 | <0.001 | S. |

Note:
N = 19; S. = Statistically significant probability (p < 0.05); L.S. = Probability close to significant (0.05 ≤ p < 0.10); N.S. = Not significant probability (p ≥ 0.10)

TABLE 20

| Time point evaluation | | Moisturization gain | Statistical Significance |
|---|---|---|---|
| T2 | hours | +42.50% | Yes |
| T4 | hours | +46.02% | Yes |
| T8 | hours | +48.13% | Yes |
| T24 | hours | +21.04% | Yes |
| T48 | hours | +12.77% | Yes |
| T72 | hours | +13.01% | Yes |

A summary of the statistical analysis of the results from the study comparing formulas 2, 4 and 5 to each other is shown in Table 21.

TABLE 21

| | | |
|---|---|---|
| | | Statistical |
| Time | Formula Comparison | Significance |

| | | | |
|---|---|---|---|
| T2 | hours | Formula 2 vs Formula 5 | N.S. |
| | | Formula 4 vs Formula 5 | N.S. |
| | | Formula 2 vs Formula 4 | N.S. |
| T4 | hours | Formula 2 vs Formula 5 | N.S. |
| | | Formula 4 vs Formula 5 | N.S. |
| | | Formula 2 vs Formula 4 | N.S. |
| T8 | hours | Formula 2 vs Formula 5 | N.S. |
| | | Formula 4 vs Formula 5 | N.S. |
| | | Formula 2 vs Formula 4 | N.S. |
| T24 | hours | Formula 2 vs Formula 5 | S. (+8.36% in favor |
| | | | of formula 5) |
| | | Formula 4 vs Formula 5 | S. (+9.15% in favor |
| | | | of formula 5) |
| | | Formula 2 vs Formula 4 | N.S. |
| T48 | hours | Formula 2 vs Formula 5 | N.S. |
| | | Formula 4 vs Formula 5 | L.S. (+8.77% in favor |
| | | | of formula 5) |
| | | Formula 2 vs Formula 4 | N.S. |
| T72 | hours | Formula 2 vs Formula 5 | S. (+6.70% in favor |
| | | | of formula 5) |
| | | Formula 4 vs Formula 5 | S. (+10.70% in favor |
| | | | of formula 5) |
| | | Formula 2 vs Formula 4 | N.S. |

Note:
S. = Statistically significant probability (p < 0.05);
L.S. = Probability close to significant (0.05 ≤ p < 0.10);
N.S. = Not significant probability (p ≥ 0.10)

In light of the results, treatment with formula 5 resulted in a better hydrating effect than the other formulas at 24 hours (T24), 48 hours (T48), and 72 hours (T72) following a single cutaneous application. Therefore, the addition of polyglutamic acid (PGA) in formula 5 reinforced the moisturizing effect over time. The differences between formulas 2 and 4 were not significant at any of the above-indicated times.

A number of different aspects and embodiments of the instant invention have been described throughout the application. Nevertheless, the skilled artisan, without departing from the spirit and scope of the instant invention, can make various changes and modifications of the instant invention to adapt it to various usages and conditions.

1. A topical composition comprising;
(i) 0.05% to 0.6% by weight allantoin;
(ii) 1.0% to 5.0% by weight Combination A, wherein Composition A comprises: 12% to 22% glycerin, 5% to 15% trehalose, 5% to 15% urea, 1% to 5% serine, 0.5% to 4% pentylene glycol, 0.10% to 2% algin or a cosmetically acceptable salt thereof, 0.1% to 0.5% glyceryl polyacrylate, 0.2% to 0.8% caprylyl glycol, 0.1% to 1.0% hyaluronic acid or a cosmetically acceptable salt thereof, 0.1% to 1.0% pullulan, and water;
(iii) 0.005% to 0.5% by weight hydrolyzed hyaluronic acid or a cosmetically acceptable salt thereof;
(iv) 0.05% to 1.0% by weight polyglutamic acid or a cosmetically acceptable salt thereof; and
(v) water.

2. The composition of claim 1, wherein said Combination A comprises by weight percent:
about 17.5% glycerin;
about 10% trehalose;
about 10% urea;
about 3% serine;
about 2% pentylene glycol;
about 0.5% sodium alginate, wherein said sodium alginate has a molecular weight of 100 to 550 kDa;
about 0.3% glyceryl polyacrylate;
about 0.5% caprylyl glycol;
about 0.25% sodium hyaluronate, wherein said sodium hyaluronate has a molecular weight of 250 to 450 kDa;
about 0.25% pullulan, wherein said pullulan has a molecular weight of 100 to 250 kDa;
about 0.1% disodium phosphate;
about 0.02% potassium phosphate; and
about 55.5% water;
said (iii) hydrolyzed hyaluronic acid or a cosmetically acceptable salt thereof is hydrolyzed hyaluronic acid; and
said (iv) polyglutamic acid or a cosmetically acceptable salt thereof is polyglutamic acid, wherein said polyglutamic acid has a molecular weight of 30 kDa to 150 kDa.

3. The composition of claim 2, further comprising:
(vi) 0.5% to 3.0% by weight Combination B, wherein Composition B comprises: xylitylglucoside, anhydroxylitol, and xylitol; and
(vii) 0.5% to 2.0% by weight Combination C, wherein Composition C comprises: 70% to 80% panthenol; and
(viii) 0.05% to 5.0% by weight Combination D, wherein Composition D comprises: glycerin, and Globularia alypum leaf extract; and
(ix) 0.25% to 3% by weight niacinamide.

4. The composition of claim 3, wherein said Combination B comprises by weight percent 1% to 25% xylitol, 15% to 35% anhydroxylitol, 35% to 80% xylitylglucoside, and water;
said Combination C comprises by weight percent about 75% pantenol; and further comprises by weight percent about 0.3% citric acid and/or about 1% D-pantolactone, and water;
said Combination D comprises by weight percent about 88% glycerin, about 6% Globularia alypum leaf extract, and about 6% water; and
said hydrolyzed hyaluronic acid or a cosmetically acceptable salt thereof is provided in an amount of 0.05% to 0.5% by weight.

5. (canceled)

6. (canceled)

7. (canceled)

8. The composition claim 4, further comprising one or more emollient A agents selected from the group consisting of dimethicone, octyldodecanol, undecane, tridecane, dicaprylyl ether, and decyl oleate, wherein said one or more emollient A agents are 2.0% to 11.0% by weight of said composition; and
one or more emollient B agents selected from the group consisting of caprylyl glycol, 1,2-hexanediol, and pentylene glycol, wherein said one or more emollient B agents are 0.1% to 2.0% by weight of said composition.

9. The composition of claim 8, wherein said one or more emollient A agents comprise low viscosity dimethicone of 4.5-5.5 centistokes at 25° C. in an amount providing 1.0% to 4.2% by weight of said composition, medium viscosity dimethicone of 332.5-367.5 centistokes at 25° C. in an amount providing 1.0% to 4.2% by weight of said composition, and octyldodecanol in an amount providing 0.5% to 4.2% by weight of said composition; and said one or more emollient B agents are by weight percent about 50% caprylyl glycol and about 50% 1,2-hexanediol.

10. (canceled)

11. (canceled)

12. The composition of claim 9, further comprising by weight percent distarch phosphate.

13. (canceled)

14. The composition of claim 12, further comprising one or more humectants selected from the group consisting of glycerin, sorbitol, and propanediol, wherein said one or more humectants is 4.0% to 15.0% by weight of said composition.

15. The composition of claim 14, wherein said one or more humectants comprises glycerin in an amount providing 2.0% to 13.0% by weight of said composition, and propanediol in an amount providing 2.0% to 13.0% weight of said composition.

16. The composition of claim 14, further comprising one or more emulsion A agents selected from the group consisting of xanthan gum, carbomer, and methylcellulose, wherein said one or more emulsion A agents is 0.1% to 2.0% by weight of said composition;
one or more emulsion B agents selected from the group consisting of carbomer, sodium acrylate/vinyl alcohol copolymer and sodium polyacryloyldimethyl taurate, wherein said one or more emulsion B agents is 0.2% to 2.0% by weight of said composition; and
one or more emulsion C agents selected from the group consisting of acrylates/C10-30 alkyl acrylate crosspolymer and carbomer, wherein said one or more emulsion C agents is 0.1% to 2.0% by weight of said composition.

17. The composition of claim 16, wherein said one or more emulsion A agents is xanthan gum,
said one or more emulsion B agents is sodium polyacryloyldimethyl taurate having a molecular weight of 450 kDa to 550 kDa; and
said one or more emulsion C agents is acrylates/C10-30 alkyl acrylate crosspolymer.

18. (canceled)

19. (canceled)

20. (canceled)

21. (canceled)

22. The composition of claim 17, further comprising one or more antioxidants selected from the group consisting of hydroxyacetophenone, pentylene glycol, and caprylyl glycol, wherein said one or more antioxidants is 0.2% to 2% by weight of said composition.

23. The composition of claim 22, wherein said one or more antioxidants is hydroxyacetophenone.

24. The composition of claim 23, further comprising a buffering agent or pH adjuster to provide a pH of 4.0 to 6.5.

25. The composition of claim 24, wherein said buffering agent or pH adjuster is sodium hydroxide.

26. The composition of claim 1, wherein said composition is either Product A, B, C, D, E, or F as provided as follows:

| | Product (about weight %) | | | | | |
|---|---|---|---|---|---|---|
| | A | B | C | D | E | F |
| Aqua | 75.68 | 73.68 | 76.77 | 77.02 | 76.77 | 75.77 |
| Glycerin | 6.0 | 7.0 | 5.0 | 6.0 | 5.0 | 6.0 |
| Propanediol | 3.0 | 4.0 | 3.0 | 3.0 | 3.0 | 3.0 |
| Distarch Phosphate | 0.50 | 0.50 | 0.50 | 2.0 | 0 | 0.5 |
| Octyldodecanol | 1.0 | 1.0 | 1.0 | 0.50 | 0.50 | 1.0 |
| Sodium | 1.0 | 1.0 | 1.0 | 1.0 | 1.0 | 1.0 |
| Polyacryloyldimethyl | | | | | | |
| Taurate | | | | | | |
| Caprylyl Glycol & | 0.50 | 0.50 | 0.50 | 0.70 | 1.0 | 0.5 |
| 1,2-Hexanediol | | | | | | |
| Hydroxyacetophenone | 0.50 | 0.50 | 0.50 | 0.50 | 0.50 | 0.5 |
| Acrylates/C10-30 | 0.30 | 0.30 | 0.30 | 0.50 | 0.50 | 0.3 |
| Alkyl Acrylate | | | | | | |
| Crosspolymer | | | | | | |
| Xanthan Gum | 0.50 | 0.50 | 0.50 | 0 | 0.30 | 0.5 |
| Sodium Hydroxide | 0.03 | 0.03 | 0.03 | 0.1 | 0.5 | 0.03 |
| Dimethicone, medium | 2.0 | 2.0 | 2.0 | 0.03 | 0.03 | 2.0 |
| viscosity, about 350 | | | | | | |
| cSt | | | | | | |
| Polyglutamic Acid | 0.10 | 0.10 | 0.10 | 1.0 | 2.0 | 0.10 |
| Combination A | 2.0 | 2.0 | 2.0 | 0.50 | 0.10 | 2.0 |
| Allantoin | 0.30 | 0.30 | 0.30 | 2.0 | 2.0 | 0.30 |
| Hydrolyzed | 0.10 | 0.10 | 0.01 | 0.50 | 0.30 | 0.01 |
| Hyaluronic Acid | | | | | | |
| Combination D | 0.15 | 0.15 | 0.15 | 0.40 | 0 | 0.15 |
| Combination C | 1.34 | 1.34 | 1.34 | 0.10 | 0.01 | 1.34 |
| Combination B | 2.0 | 2.0 | 2.0 | 0.15 | 0.15 | 2.0 |
| Dimethicone low | 2.0 | 2.0 | 2.0 | 1.0 | 1.34 | 2.0 |
| viscosity about 5 cSt | | | | | | |
| Niacinamide | 1.0 | 1.0 | 1.0 | 2.0 | 2.0 | 1.0 |

wherein Combination A comprises by weight percent; about 17.5% glycerin; about 10% trehalose; about 10% urea; about 3% serine; about 2% pentylene glycol; about 0.5% algin or a cosmetically acceptably salt thereof, having a molecular weight of 100 to 550 kDa, about 0.3% o glyceryl polyacrylate; about 0.5% caprylyl glycol; about 0.25% hyaluronic acid or a cosmetically acceptable salt thereof, having a molecular weight of 250 to 450 kDa; about 0.25% pullulan, having a molecular weight of 100 to 250 kDa; about 55.500 water, about 0.1% disodium phosphate and about 0.02% potassium phosphate;
Combination B comprises by weight percent 1% to 25% xylitol, 15% to 35% anhydroxylitol, 3500 to 80% xylitylglucoside, 300 to 20% xylitol, 20% to 30% anhydroxylitol, 40% to 77% xylitylglucoside and 15% to 17% water;
Combination C comprises by weight percent about 7500 pantenol, about 0.3% citric acid and about 1% D-pantolactone, and water;
Combination D comprises by weight percent about 88% glycerin, about 6% Globularia alypum leaf extract and about 6% water; and
said composition has a pH of 4.0 to 6.5.

27. The composition of claim 26, wherein said composition is an oil-free gel.

28. (canceled)

29. A method of treating human skin comprising the step of applying an effective amount of the composition of claim 26 to the skin.

30. (canceled)

31. The method of claim 29, wherein said skin is dry skin and said method applies an effective amount of the composition to treat the dry skin for greater than 48 hours, and/or said skin is sensitive skin and said method comprises applying an effective amount of the composition to treat the sensitive skin for greater than 48 hours.

32. (canceled)

33. (canceled)

34. (canceled)

35. (canceled)

36. The method of claim 29, wherein said composition is used as a facial mask for treating dry skin comprising the step of applying an effective amount of the composition as a mask on the face 2 to 3 times a week for 10 to 20 minutes each time.
